(19) United States
(12) Patent Application Publication
(10) Pub. No.: US 2026/0166099 A1
(43) Pub. Date: Jun. 18, 2026

(54) COMPOSITIONS AND METHODS RELATED TO THE USE OF BACTERIA TO REMOVE PLAQUE, FATS, AND LIPIDS FROM HUMAN PATIENTS

(71) Applicant: Sean Anthony Cruz, Ogden, UT (US)

(72) Inventor: Sean Anthony Cruz, Ogden, UT (US)

(21) Appl. No.: 18/979,034

(22) Filed: Dec. 12, 2024

(51) Int. Cl. A61K 35/742; A61K 9/00; A61K 31/519; A61K 35/00; A61P 3/04

(52) U.S. Cl. CPC A61K 35/742; A61K 9/0019; A61K 31/519; A61P 3/04; A61K 2035/115

(57) ABSTRACT

Various aspects of this disclosure relate to the administration of a pharmaceutical composition comprising bacteria to a human patient to kill adipocytes, mobilize fat, and/or reduce adipose tissue. The bacteria may be administered, for example, by injection into adipose tissue, a lipoma, or a liposarcoma. The bacteria may be selected or engineered to result in a self-limiting infection such that the immune system clears the infection and/or an antibiotic may be administered following administration of the pharmaceutical composition to help clear the infection.

### BACKGROUND

The removal of diseased cells and tissue from human patients is becoming increasingly sophisticated. Contemporary methods include excisional surgery, laparoscopic surgery, brachytherapy, chemotherapy, immunotherapy, cryotherapy, hyperthermia, electrodesiccation and curettage, radiofrequency ablation, and laser therapy. Medical researchers also occasionally study the use of bacteria to target and destroy tumors. See, e.g., Nguyen et al., NATURE COMMUNICATIONS, 2023, 14: 3553. Bacteria present an attractive therapeutic intervention, for example, because their relatively-simple genomes are easy to engineer, they are inexpensive to culture, and they can target tumors with multiple synergistic mechanisms such as by simultaneously secreting toxins, enhancing the immunogenicity of a tumor, and releasing cytokines to recruit an immune response. Innovative methods to remove unwanted cells and tissue nevertheless remain desirable.

### SUMMARY

Various aspects of this disclosure relate to the use of bacteria to remove plaque, fats, and lipids from human patients. Bacterial-based therapeutics present an emerging area of research for treating tumors. Mechanisms of action include direct tumor destruction, immune system activation, and the delivery of cytotoxic agents and/or immunostimulatory cytokines. Bacteria may also be bioengineered to inhibit collateral damage to healthy cells and to express therapeutic agents that enhance tumor targeting and killing as well as the recruitment and activation of immune cells. This disclosure relates to the use of bacteria to instead remodel plaque, fats, and lipids to treat one or more of obesity, atherosclerosis, lipomas, liposarcomas, and related conditions.

Researchers at Johns Hopkins University developed a Clostridium novyi therapeutic that they licensed to Biomed Valley Discoveries, which recently began clinical trials in cancer patients who failed to respond to conventional interventions. Another team at Actym Therapeutics recently announced clearance to begin clinical to assess the safety and efficacy of Salmonella typhimurium to treat solid tumors in patients who failed prior lines of therapy and lack other treatment options. Examples of bacterial-based therapeutics are described, for example, in U.S. Pat. Nos. 10,729,731, 11,168,326, 11,219,671, 11,576,936, 11,723,932, 11,779,612, 12,012,600, 12,024,709, U.S. Patent Application Publication No. 2017/0020931 A1, U.S. Patent Application Publication No. 2022/0135980A1 , U.S. Patent Application Publication No. 2022/0380720A1 , and U.S. Patent Application Publication No. 2023/0346851 A1, each of which is incorporated by reference in its entirety.

This disclosure relates to the use of bacterial-based therapeutics to target adipocytes, lipids, and atheromatous plaque instead of tumors. The bacterial-based therapies of this disclosure differ from those in development to treat tumors because (1) they are generally not administered in combination with antineoplastic agents, and (2) they generally lack bioengineered immunomodulatory features such as the ability to express immunostimulatory cytokines. Current therapeutic interventions to remodel and remove fat include liposuction, endoscopic subcutaneous fat removal, deoxycholic acid injection, high-intensity focused ultrasound, laser lipolysis, and cryolipolysis, which display varying risk/benefit profiles. In particular, the deoxycholic acid injection KYBELLA® was approved by the United States Food and Drug Administration (FDA) to remove submental fat that presents as a “double chin.” KYBELLA® injections kill adipocytes, and the body slowly mobilizes lipids from the adipocytes to permanently reduce submental fat. Analogous to KYBELLA®, the administration of bacteria of this disclosure may also kill adipocytes, mobilize fat, and permanently reduce adipose tissue.

This disclosure relates to the administration of bacteria to a human patient such as by injection to kill adipocytes, mobilize lipids, and/or reduce atheromatous plaque. The bacteria may be optionally administered with an immunosuppressant to at least partially attenuate an immune response against the bacteria. Following administration, the bacteria metabolizes lipids and/or kills adipocytes to remodel its target. The bacteria may optionally be replication deficient in vivo such that the infection is self-limiting and/or an antibiotic may be administered to clear the infection following administration. Replication deficiency and/or antibiotic sensitivity may be genetically engineered, or bacterial strains may be selected that display inherent replication deficiency and/or antibiotic sensitivity in humans.

The foregoing features and variations thereof will be readily apparent to those of ordinary skill in the art. Neither this Summary section, nor the foregoing Background, nor the following Detailed Description and Exemplification shall limit any patent claim that matures from this disclosure, which patent claim(s) shall instead be construed first in accordance with their literal scope and the context of their claim dependency, and then, if any ambiguity exists, in accordance with the canons of claim construction.

### DETAILED DESCRIPTION

Various aspects of this disclosure relate to a method to administer bacteria to a human patient, comprising providing a pharmaceutical composition comprising the bacteria and administering the pharmaceutical composition to the patient. In some embodiments, the pharmaceutical composition comprises a single strain of bacteria. In some specific embodiments, the pharmaceutical composition comprises a single strain of bacteria and lacks any other strain of bacteria.

In some embodiments, the patient presents with one or more of unwanted body fat, unwanted body fat distribution, a body mass index (BMI) of at least 25, a BMI of at least 30, obesity, atheromatous plaque, atherosclerosis, a lipoma, or a liposarcoma. In some embodiments, the patient does not have a tumor. In some embodiments, the patient does not have cancer.

In some embodiments, the bacteria is selected from Acinetobacter, Aeromonas, Agrobacterium, Alcaligenes, Alteromonas, Bacillus, Bifidobacterium, Bordetella, Borrelia, Brucella, Chlamydia, Citrobacter, Clostridium, Corynebacterium, Enterobacter, Erysipelothrix, Escherichia, Flavobacterium, Francisella, Heliobacter, Hemophilus, Klebsiella, Listeria, Micrococcus, Moraxella, Mycobacterium, Mycoplasma, Neisseria, Pseudomonas, Rhodococcus, Rochalimaea, Rikettsia, Salmonella, Serratia, Shigella, Staphylococcus, Streptococcus, Treponema, Vibrio, and Yersinia. See, e.g., U.S. Pat. Nos. 6,190,657, 6,863,894, 6,923,972, 7,452,531, 9,181,546, 10,987,432, 11,103,538, 11,141,492, 11,168,326, U.S. Patent Application Publication No. 2003/0175297A1 , U.S. Patent Application Publication No. 2007/0298012A1 , and U.S. Patent Application Publication No. 2012/0009153A1 , each of which is incorporated by reference in its entirety.

In some embodiments, the bacteria is a gram-positive bacteria.

In some embodiments, the bacteria is selected from Acinetobacter, Alcaligenes, Alteromonas, Bacillus, Bifidobacterium, Clostridium, Enterobacter, Flavobacterium, Listeria, Micrococcus, Moraxella, Mycobacterium, Salmonella, Serratia, Staphylococcus, Streptococcus, and Yersinia.

In some embodiments, the bacteria is selected from Acinetobacter baumannii, Acinetobacter baylyi, Acinetobacter lwoffii, Acinetobacter calcoaceticus, Aeromonas eucrenophila, Alcaligenes faecalis, Alteromonas addita, Alteromonas genovensis, Alteromonas hispanica, Alteromonas litorea, Alteromonas macleodii, Alteromonas marina, Alteromonas simiduii, Aeromonas salminocida, Agrobacterium tumerfacium, Alteromonas stellipolaris, Alteromonas tagae, Bacillus anthracis, Bacillus licheniformis, Bacillus subtilis, Bifidobacterium bifidum, Bordetella bronchiseptica, Borrelia burgdorferi, Brucella abortus, Brucella melitensis, Chlamydia pneumoniae, Citrobacter freundii, Clostridioides difficile, Clostridium histolyticus, Clostridium novyi, Corynebacterium pseudotuberculosis, Cryptococcus neoformans, Eimeria acervulina, Encephahtozoon cuniculi, Enterobacter cloacae, Erysipelothrix rhusiopathiae, Escherichia coli, Flavobacterium petrolei, Flavobacterium psychrolimnae, Franciesella tularensis, Heliobacter mustelae, Hemophilus sornnus, Histoplasma capsulatum, Leishmania amazonensis, Leishmania major, Leishmania mexacana, Legionella pneumophila, Leptomonas karyophilus, Listeria monocytogenes, Micrococcus luteus, Moraxella bovis, Moraxella catarrhalis, Moraxella lacunata, Mycobacterium avium, Mycobacterium bovis, Mycobacterium indicus pranii, Mycobacterium intracellulare, Mycobacterium tuberculosis, Mycoplasma hominis, Neisseria gonorrhoeae, Neisseria meningitidis, Neospora caninum, Nosema helminthorum, Plasmodium falciparum, Pneumocystis carnii, Pseudomonas aeruginosa, Rhodococcus equi, Rickettsia mooseri, Riketsia prowaseckii, Rickettsiae quintana, Rickettsia sibirica, Rickettsia tikettsiae, Rickettsia tsutsugamuchi, Rochalimaea quitana, Salmonella choleraesuis, Salmonella enterica, Salmonella enteritidis, Salmonella galinarum, Salmonella typhi, Salmonella typhimurium, Sarcocystis suihominis, Shigella disenteriae, Shigella flexneri, Shigella sonnei, Staphylococcus aureus, Staphylococcus epidermidis, Streptococcus gordonii, Streptococcus pyogenes, Treponema pallidum, Trypanasoma cruzi, Unikaryon legeri, Vibrio cholera, Yersinia enterocolitica, and Yersinia pestis.

In some specific embodiments, the bacteria is selected from Alteromonas, Bacillus, Flavobacterium, Micrococcus, Pseudomonas, and Staphylococcus. In some very specific embodiments, the bacteria is selected from Alteromonas addita, Alteromonas genovensis, Alteromonas hispanica, Alteromonas litorea, Alteromonas macleodii, Alteromonas marina, Alteromonas simiduii, Bacillus anthracis, Bacillus licheniformis, Bacillus subtilis, Flavobacterium petrolei, Flavobacterium psychrolimnae, Micrococcus luteus, Pneumocystis carnii, Pseudomonas aeruginosa, Staphylococcus aureus, and Staphylococcus epidermidis.

In some embodiments, the bacteria is attenuated. Auxotrophic mutations, for example, attenuate bacteria by inhibiting the biosynthesis of an essential nutrient. In general, auxotrophic mutations impair the ability of bacteria to survive in the absence of the essential nutrient. Salmonella and other species can be attenuated, for example, by deleting the aroA, aroC, and/or aroD genes, which impairs the biosynthesis of one or more aromatic amino acids. Salmonella typhimurium strain SL7207, for example, lacks a functional aroA gene. Other mutations can impair the biosynthesis of different amino acids such as leucine or arginine. Other auxotrophic mutations inhibit the biosynthesis of nucleosides, which impairs the ability to produce nucleic acid building blocks and even the production of adenosine triphosphate (ATP), the fundamental energy currency of the cell. Salmonella typhimurium strain VNP20009 lacks a functional purI gene, for example, and cannot synthesize the nucleoside adenosine. Other attenuating mutations inhibit virulence factors or disrupt the cell cycle. Various attenuating mutations are described in the patents and patent application publications that are cited herein and incorporated by reference.

In some embodiments, the bacteria expresses an extracellular lipase. In some specific embodiments, the bacteria is genetically modified to express the extracellular lipase. Lipases hydrolyze fats, and certain bacteria that secrete lipases are more effective at mobilizing fats. In some very specific embodiments, the extracellular lipase is effective to hydrolyze triglycerides of the patient.

In some embodiments, the bacteria produces deoxycholic acid. Some bacteria such as Clostridium naturally produce deoxycholic acid, and bacteria may also be genetically modified to produce deoxycholic acid. In some specific embodiments, the bacteria is genetically modified to produce deoxycholic acid. In some very specific embodiments, the deoxycholic acid is effective to mobilize fat in the human patient.

In some embodiments, the administering is injecting or infusing. In some specific embodiments, the administering is performed by subcutaneous injection, intravenous injection, or intraarterial. In some very specific embodiments, the administering is performed by injection into or adjacent to adipose tissue or a non-carcinogenic tumor such as a lipoma. In some specific embodiments, the patient presents with a lipocarcinoma, and the method comprises injecting the pharmaceutical composition into the lipocarcinoma.

In some embodiments, the administering comprises at least one injection such as at least one subcutaneous injection into or adjacent to adipose tissue or a non-carcinogenic tumor. The administering may be performed, for example, by injecting the pharmaceutical composition at various different positions on the body such as at several different positions of the panniculus adiposus for patients who present with such.

In some embodiments, the pharmaceutical composition is effective to mobilize body fat in the patient.

In some embodiments, the administering is to adipose tissue, and the bacteria is effective to hydrolyze triglycerides of the adipose tissue.

In some embodiments, the patient presents with excess body fat, and the administering comprises injecting the pharmaceutical composition into the excess body fat.

In some embodiments, the patient presents with atherosclerosis, and the administering is intravenous or intraarterial injecting.

In some embodiments, the patient presents with atheromatous plaque, and the bacteria is effective to mobilize the atheromatous plaque in the human patient.

In some embodiments, the pharmaceutical composition is formulated for injection or infusion. In some specific embodiments, the pharmaceutical composition is formulated as a suspension of the bacteria in water. In some very specific embodiments, the pharmaceutical composition is formulated as a suspension of bacteria in water, and the water comprises one or more dissolved solutes. The dissolved solutes may include one or more metal cations (such as sodium cation, potassium cation, calcium cation, magnesium cation, iron(II), iron(III), manganese(II), and zinc(II)), counterions (such as chloride ion and sulfate), buffer (such as dihydrogen phosphate and hydrogen phosphate or mono-hydrogen citrate and citrate), carbon source (such as glucose), and nitrogen source (such as ammonium). Various formulations and their methods of manufacture are described in the patents and patent application publications that are cited herein and incorporated by reference. General considerations for formulating compositions of this disclosure may also be found, for example, in REMINGTON: THE SCIENCE AND PRACTICE OF PHARMACY, 22nd edition (Allen Jr, Loyd V., editor) Pharmaceutical Press, 2012.

In some embodiments, the pharmaceutical composition comprises about 100 thousand to about one trillion colony forming units (CFUs) of the bacteria. In some specific embodiments, the pharmaceutical composition comprises about one million to about one billion CFUs of the bacteria.

In some embodiments, the administering comprises administering about 100 thousand to about one trillion CFUs of the bacteria to the patient. In some specific embodiments, the pharmaceutical composition comprises about one million to about one billion CFUs of the bacteria to the patient.

In some embodiments, the method comprises administering an immunosuppressant to the patient, wherein the immunosuppressant is effective to inhibit an immune response by the patient against the bacteria. Suitable immunosuppressants are known and include, for example, anti-CD20 antibodies, anti-CD3 antibodies, anti-CD25 antibodies, azathioprine, bleomycin, ciclosporin, dactinomycin, daunorubicin, doxorubicin, epirubicin, everolimus, fingolimod, fluorouracil, glucocorticoids, idarubicin, interferon beta, mercaptopurine, methotrexate, mitomycin C, myriocin, plicamycin, sirolimus, tacrolimus, and zotarolimus. The routes of administration and dosing of such immunosuppressants are known and can be readily identified by those skilled in the art, for example, by reviewing the FDA labels of the foregoing.

In some embodiments, the method comprises administering the immunosuppressant prior to administering the pharmaceutical composition. Administering the immunosuppressant prior to administering the pharmaceutical composition may be more effective at inhibiting acute immune responses against the bacteria, for example, relative to co-administering the immunosuppressant with the pharmaceutical composition. In some specific embodiments, the method comprises administering the immunosuppressant six hours to one week prior to administering the pharmaceutical composition. The method may nevertheless comprise administering multiple doses of the immunosuppressant such as an initial dose prior to administering the pharmaceutical composition, a subsequent dose on the same day that the pharmaceutical composition is administered, and/or a final does a period of time after the pharmaceutical composition is administered. The period of time after the pharmaceutical composition is administered is dependent upon the properties of a specific immunosuppressant including, for example, its half-life and duration of action, and exemplary periods of time range from one to 30 days subsequent to the administration of the pharmaceutical composition such as one to 14 days or one to eight days.

In some embodiments, the method comprises administering an antibiotic to the patient a period of time after administering the antibody, wherein the antibiotic is effective to kill the bacteria. Those skilled in the art will immediately recognize suitable antibiotics, which necessarily must be selected in view of the bacteria. Beta-lactam antibiotics such as cephalosporins (e.g., cefalexin) are generally effective against gram-positive bacteria (e.g., Clostridium) as well as some gram-negative bacteria. Aminoglycoside antibiotics such as gentamicin are generally effective against gram-negative bacteria (e.g., Escherichia, Klebsiella, and Pseudomonas) as well as some gram-positive bacteria (e.g., Staphylococcus). The route of administration may be, for example, systemic such as oral or intravenous and/or local such as by injection at or adjacent to a site of injection of the pharmaceutical composition.

In some embodiments, the method comprises administering the pharmaceutical composition by injection (such as by injection into or adjacent to adipose tissue) and marking the site(s) of injection (such as by encircling the site(s) with a permanent marker before or after administering the composition). In some specific embodiments, the method comprises administering the pharmaceutical composition by injection, marking the site(s) of injection, and then injecting the antibiotic at the marked site(s) of injection (such as within the region(s) encircled with the permanent marker).

In some embodiments, the period of time between the administration of the pharmaceutical composition and the administration of the antibiotic is six hours to three weeks such as nine hours to 14 days or twelve hours to seven days.

In some embodiments, the method comprises monitoring one or more of the heart rate, blood pressure, and body temperature of the patient subsequent to administering the pharmaceutical composition. Side effects of administering the bacteria include, for example, a risk of sepsis and/or a deleterious immune response such as an allergic reaction that in some rare instances may result in anaphylaxis. Increased heart rate, blood pressure, and/or body temperature following the administration of the pharmaceutical composition may be predictive of the risk of sepsis and/or anaphylaxis. If one or more of heart rate, blood pressure, and body temperature indicates such a risk, then one or more of antibiotics, antihistamines, glucocorticoids, epinephrine, anticholinergics, mast cell stabilizers, and antileukotriene agents may be administered to the patient to inhibit the risk of serious side effects.

In some embodiments, the method comprises administering an inactivated composition comprising an inactivated bacteria prior to administering the pharmaceutical composition, wherein the inactivated bacteria is a dead form of the bacteria. The inactivated bacteria may be killed, for example, by chemicals (such as ethanol), heat, or radiation. The inactivated composition may be administered, for example, to determine whether the patient displays a deleterious allergic reaction to the bacteria. Administering the pharmaceutical composition might only be suitable for a given patient if the inactivated composition does not display a deleterious allergic reaction in the given patient. The inactivated composition may also be administered, for example, to develop a primary immune response against the bacteria such that the patient can clear bacteria of the pharmaceutical composition such as after immunosuppressants are discontinued.

### EXEMPLIFICATION

Example 1. A single injection of Bacillus licheniformis into obese, BALB/c mice decreases visceral body fat, reduces appetite, and reduces body weight.

Four-week-old, male, BALB/c nude mice are obtained from The Jackson Laboratory (Maine, United States) and fed a high-fat diet ad libitum for eight weeks to induce obesity (40% kcal from fat, 34% carbohydrate, 26% protein). Nude mice comprise a deficient immune system as a result of deletion of the FOXN1 gene, which is required to form a functional thymus, and they were selected for initial experiments to avoid an immune response against bacterial-based therapeutics.

At twelve weeks, 10E5 CFU, 10E6 CFU, or 10E7 CFU of B. licheniformis is injected into the visceral adipose tissue on the left side of five mice per treatment group, and vehicle is injected into five control mice. During the four weeks post treatment, the treated mice consume significantly less chow than the control mice. The mice are monitored for four weeks post treatment and then sacrificed.

B. licheniformis secretes enzymes at body temperature (37 ° Celsius), which may be beneficial for remodeling adipose tissue. B. licheniformis is readily propagated culture and presents with a long history of commercial use (e.g., to manufacture various enzymes). B. licheniformis presents a low risk to humans, a low risk of transmission between humans, and infections are generally self-limiting. Any infection may nevertheless be cleared with cephalosporins (e.g., cefalexin, cefepime, cefuroxime), carbapenems (e.g., doripenem, imipenem, meropenem), aminoglycosides (e.g., gentamicin, tobramycin, amikacin, neomycin, plazomicin, streptomycin), or vancomycin either alone or in combination.

The mice weigh 30 grams at twelve weeks (±2 grams). Four weeks post treatment, the mice that received the bacteria display a dose-dependent decrease in body weight, and the mice that received vehicle increase their body weights to 32 grams (±2 grams). The visceral adipose tissue of the mice is excised and weighed, and the weight of the visceral adipose tissue of the treated mice is approximately 20% less than the control mice. Histology of the adipose tissue from the left and right sides of the abdomen of the treated mice show marked differences consistent with the death of adipocytes on the left side and mobilization of triglycerides.

This experiment suggests that treating mice with the B. licheniformis mobilizes fat from visceral adipose tissue, reduces appetite, and ultimately reduces body weight.

Example 2. A single injection of Bacillus licheniformis into obese mice decreases visceral body fat, reduces appetite, and reduces body weight.

Eight-week-old male B6.V-Lep^{ob}/J mice are obtained from The Jackson Laboratory and fed chow ad libitum. These mice display a type 2 diabetes phenotype and severe obesity. Each mouse weighs approximately 40 grams (±1 gram) at eight weeks.

Each mouse is administered methotrexate daily at 10 milligrams per kilogram body weight to attenuate its immune system, which is continued until the mice are sacrificed. Following one week of methotrexate treatment, 10E5 CFU, 10E6 CFU, or 10E7 CFU of B. licheniformis is injected into the visceral adipose tissue on the left side of five mice per treatment group, and vehicle is injected into five control mice. The mice are monitored for four weeks post treatment and then sacrificed. During the four weeks post treatment, the treated mice consume significantly less chow than the control mice.

The mice weigh 45 grams at nine weeks (±2 grams). Four weeks post treatment, the mice that received the bacteria display a dose-dependent decrease in body weight, and the mice that received vehicle increase their body weights to 50 grams (±2 grams). The visceral adipose tissue of the mice is excised and weighed, and the weight of the visceral adipose tissue of the treated mice is approximately 25% less than the control mice. Histology of the adipose tissue from the left and right sides of the abdomen of the treated mice show marked differences consistent with the death of adipocytes on the left side and mobilization of triglycerides.

This experiment suggests that treating mice with the B. licheniformis mobilizes fat from visceral adipose tissue, reduces appetite, and ultimately reduces body weight.

What is claimed is:

1. A method to administer bacteria to a human patient, comprising:
providing a pharmaceutical composition comprising a single strain of the bacteria; and
administering the pharmaceutical composition to the blood or to the adipose tissue of the human patient.

2. The method of claim 1, wherein the administering is injecting.

3. The method of claim 2, wherein the injecting is intravenous injecting.

4. The method of claim 2, wherein the injecting is subcutaneous injecting.

5. The method of claim 1, comprising administering an immunosuppressant to the human patient, wherein the immunosuppressant is effective to inhibit an immune response by the human patient against the bacteria.

6. The method of claim 5, wherein the immunosuppressant is selected from an anti-CD20 antibody, an anti-CD3 antibody, an anti-CD25 antibody, azathioprine, bleomycin, ciclosporin, dactinomycin, daunorubicin, doxorubicin, epirubicin, everolimus, fingolimod, fluorouracil, a glucocorticoid, idarubicin, interferon beta, mercaptopurine, methotrexate, mitomycin C, myriocin, plicamycin, sirolimus, tacrolimus, and zotarolimus.

7. The method of claim 1, comprising administering an antibiotic to the human patient a period of time after administering the antibody, wherein the antibiotic is effective to kill the bacteria.

8. The method of claim 7, wherein the period of time is twelve hours to seven days after administering the pharmaceutical composition.

9. The method of claim 1, wherein the bacteria is replication deficient in the human patient.

10. The method of claim 1, comprising monitoring the heart rate and the body temperature of the human patient following the administering.

11. The method of claim 1, wherein the bacteria expresses an extracellular lipase.

12. The method of claim 11, wherein the bacteria is genetically modified to express the extracellular lipase.

13. The method of claim 11, wherein the extracellular lipase is effective to hydrolyze triglycerides of the human patient.

14. The method of claim 1, wherein the pharmaceutical composition is effective to mobilize body fat in the human patient.

15. The method of claim 1, wherein the administering is to adipose tissue, and the bacteria is effective to hydrolyze triglycerides of the adipose tissue.

16. The method of claim 1, wherein the human patient presents with excess body fat, and the administering comprises injecting the pharmaceutical composition into the excess body fat.

17. The method of claim 1, wherein the human patient presents with atheromatous plaque, and the bacteria is effective to mobilize the atheromatous plaque in the human patient.

18. The method of claim 1, wherein the human patient presents with atherosclerosis, and the administering is intravenous or intraarterial injecting.

19. The method of claim 1, wherein the bacteria is a gram-positive bacteria.

20. The method of claim 1, wherein the bacteria is selected from Alteromonas, Bacillus, Flavobacterium, Micrococcus, Pseudomonas, and Staphylococcus.
